(19) United States
(12) Patent Application Publication
(10) Pub. No.: US 2023/0382939 A1
(43) Pub. Date: Nov. 30, 2023

(54) MACROCYCLIC HOST MOLECULE-BASED CORE-SHELL NANOPARTICLES AND METHOD FOR SYNTHESIZING SAME

(71) Applicants: SEOUL NATIONAL UNIVERSITY R&DB FOUNDATION, Seoul (KR); BIONANO HEALTH GUARD RESEARCH CENTER, Daejeon (KR)

(72) Inventors: Jwa-Min NAM, Seoul (KR); Jae-Myoung KIM, Seoul (KR); Jiyeon KIM, Seoul (KR)

(21) Appl. No.: 18/246,944

(22) PCT Filed: Sep. 6, 2021
(86) PCT No.: PCT/KR2021/012021

§ 371 Date: Aug. 23, 2023

(30) Foreign Application Priority Data
Sep. 28, 2020 (KR) 10-2020-0126084

(51) Int. Cl. C07H 5/10; C07C 211/62; C01F 17/271; C07D 307/33

(52) U.S. Cl. CPC C07H 5/10; C07C 211/62; B82Y 40/00; C07D 307/33; C01F 17/271

(57) ABSTRACT

Provided is a nanoparticle including: a core structure; and a shell structure covering the core structure and separated from the core structure by a nanogap, wherein a macrocyclic host molecule exhibiting hydrophobicity inside and hydrophilicity outside and a Raman-active material inserted into the macrocyclic host molecule are provided on a surface of the core structure, and the macrocyclic host molecule and the Raman-active material fill the nanogap.

### TECHNICAL FIELD

The present invention relates to macrocyclic host molecule-based core-shell nanoparticles and a method for synthesizing same.

### BACKGROUND ART

Highly sensitive, accurate detection of single molecules from biological samples or other samples may be widely used in many fields such as medical diagnosis, pathology, environmental sampling, and chemical analysis. To this end, the biology-chemistry field has widely utilized specifically labeled nanoparticles or chemical materials in studying the metabolism, distribution, and binding of small synthetic materials and biomolecules. Typically, methods of using radioactive isotopes, organic fluorescent dyes, and inorganic materials such as quantum dots have been used.

In methods of using radioactive isotopes such as ³H, ¹⁴C, ³²P, ³⁵S, ¹²⁵I, etc., which are radioactive isotopes of ¹H, ¹²C, ³¹P, ³²S, ¹²⁷I, etc. widely distributed in the body, have been widely used. Radioactive isotopes have been used for a long time because radioactive and non-radioactive isotopes may be used interchangeably due to almost the same chemical properties, and because even a small amount of radioactive isotopes may be detected due to their relatively high emission energy. However, radioactive isotopes are difficult to handle because the radiation produced thereby is harmful to the body. Additionally, although their emission energy is high, some of the radioactive isotopes have short half-lives so that they cannot be stored for a long period of time or are not suitable for use in long-term experiments.

Organic fluorescent dyes have been widely used as an alternative to radioactive isotopes. The organic fluorescent dyes are activated by absorbing energy of a specific wavelength and emit light at a certain wavelength. Particularly, as detection methods become simplified, radioactive substances face problems with detection limits and thus require long periods of time for detection. In contrast, an organic fluorescent dye theoretically allows the detection of even in a single molecular level because thousands of photons per molecule may be emitted thereby under the proper conditions. However, unlike radioactive isotopes, fluorescent dyes cannot substitute for elements of active ligands directly. Instead, they are restrictively designed to be linked to moieties which have no effects on activity in light of the structure activity relationship. In addition, these fluorescent dyes have disadvantages of photobleaching over time and interference with other fluorescent dyes due to a very narrow wavelength range of light for activation and a very wide wavelength range of emitted light. Moreover, only a small number of fluorescent dyes is available.

A quantum dot that is a semiconductor nanomaterial is composed typically of CdSe, CdS, ZnS, ZnSe, or the like, and emits light of different colors depending on size of particles and type. Compared to organic fluorescent dyes, quantum dots may be excited with a wider spectrum of excitation wavelength, emit light in a narrower spectrum of wavelengths, and thus a larger number of different colors are emitted thereby. Accordingly, quantum dots have attracted a lot of attention due to their advantages over organic fluorescent dyes. However, quantum dots have disadvantage of being highly toxic and being difficult to produce on a large scale. In addition, the number of available quantum dots, although theoretically variable, is highly restricted in practice.

To overcome such problems, labeling materials using Raman Spectrometry and/or Surface Plasmon Resonance have been recently used. Surface Enhanced Raman Scattering (SERS) is a spectroscopic method that utilizes a phenomenon whereby the intensity of Raman scattering is dramatically increased by 10⁶ to 10⁸ times or more when molecules are adsorbed on a roughened surface of a metal nanostructure such as gold or silver nanoparticles. As light passes through a transparent medium, a small fraction of the light is deviated from its original direction, known as Raman scattering. A fraction of the scattered light is excited to higher energy levels, and the scattered light has a frequency different from that of the incident light. Because the wavelengths of the Raman scattering spectrum reflect the chemical compositions and structural properties of the light absorbing molecules in a sample, Raman spectroscopy, together with the nanotechnology that is currently being developed, may be further developed for high sensitive detection of a single molecule, particularly, there is a strong expectation that a SERS sensor may be used importantly as a medical sensor. The SERS effect is in relation with Plasmon resonance. In this regard, metal nanoparticles exhibit distinct optical resonance in response to the incident electromagnetic radiation due to collective coupling of conduction electrons within the metal. Thus, nanoparticles of gold, silver, copper and other specific metals may fundamentally serve as nanoscale antenna for amplifying the localization of electromagnetic radiations. Molecules localized in the vicinity of these particles show far greater sensitivities to Raman spectroscopy.

Accordingly, in addition to highly sensitive DNA analysis, many studies are being intensively carried out about using SERS sensors to detect genes and proteins (biomarkers) for early diagnosis of various diseases. Raman spectroscopy has various advantages over other methods (infrared spectroscopy). In contrast, infrared spectroscopy may detect strong signals from molecules which have a dipole moment, Raman spectroscopy allows strong signals to be detected even from non-polar molecules in which induced polarizability is modulated. Hence, almost all organic molecules have their own Raman shifts (cm⁻¹). In addition, being free from the interference of water molecules, Raman spectroscopy is suitable for use in the detection of biomolecules such as proteins and genes. However, due to low signal intensity, the development of Raman spectroscopy has not yet reached the level where it can be used in practice in spite of research conducted for a significantly long period of time.

### DISCLOSURE

### Technical Problem

An object of the present invention is to provide a method for synthesizing nanoparticles having uniform nanogaps with a high yield.

Another object of the present invention is to provide nanoparticles enabling sensing and imaging with ultra-high sensitivity.

### Technical Solution

According to an embodiment of the present invention, provided is a nanoparticle including: a core structure; and a shell structure covering the core structure and separated from the core structure by a nanogap, wherein a macrocyclic host molecule exhibiting hydrophobicity inside and hydrophilicity outside and a Raman-active material inserted into the macrocyclic host molecule are provided on a surface of the core structure, and the macrocyclic host molecule and the Raman-active material fill the nanogap.

According to an embodiment of the present invention, the macrocyclic host molecule may include at least one compound selected from the group consisting of cyclodextrin, cucurbituril, calixarene, and pillararene and a derivative thereof.

According to an embodiment of the present invention, the macrocyclic host molecule may bind to the Raman-active material by a non-covalent bond.

According to an embodiment of the present invention, the core structure and the shell structure may include at least one material selected from gold, silver, and copper.

According to an embodiment of the present invention, the nanogap may have a size of 0.1 nm to 10 nm.

According to an embodiment of the present invention, provided is a method for synthesizing a nanoparticle including: a first step of attaching a macrocyclic host molecule onto a surface of a core structure to form a modified core structure; a second step of inserting a Raman-active material into the macrocyclic host molecule by mixing the modified core structure with the Raman-active material; and a third step of synthesizing a shell structure on the surface of the modified core structure inserted with the Raman-active material, wherein a nanogap filled with the macrocyclic host molecule and the Raman-active material is provided between the core structure and the shell structure.

According to an embodiment of the present invention, the first step may be performed by mixing the core structure with the macrocyclic host molecule and incubating the mixture, and the macrocyclic host molecule may include at least one compound selected from the group consisting of cyclodextrin, cucurbituril, calixarene, and pillararene and a derivative thereof.

According to an embodiment of the present invention, the first step may further include: a step of providing a core structure capped with cetyltrimethylammonium bromide (CTAB); and a step of providing a core structure capped with cetyltrimethylammonium chloride (CTAC) by reacting the CTAB-capped core structure with L-ascorbic acid and HAuCl₄, wherein the macrocyclic host molecule is attached onto the CTAC-capped core structure to form the modified core structure .

### Advantageous Effects

According to the method of the present invention for synthesizing nanoparticles, inner gaps may be uniformly formed with a high yield and a Raman-active material may be introduced into nanogap, by using host-guest interactions.

The nanoparticles of the present invention exhibit strong and narrow distribution of SERS enhancement factor (EF) and ultra-high sensing and imaging is possible using the same. Furthermore, it is possible to simultaneously detect various substances to be detected by using different and distinguishable detection substances.

### BRIEF DESCRIPTION OF DRAWINGS

FIG. 1 is a cross-sectional view of a nanoparticle according to an embodiment of the present invention.

FIG. 2 is a flowchart illustrating a method for synthesizing nanoparticles according to an embodiment of the present invention.

FIG. 3 shows graphs and images illustrating synthesis of macrocyclic host molecule-based nanoparticles according to an embodiment of the present invention and analysis results thereof.

FIG. 4 is a transmission electron microscope (TEM) image of core structures having a size of 50 nm on a scale of 100 nm.

FIG. 5 is an HAADF-STEM image of nanoparticles on a scale of 200 nm.

FIG. 6 shows HAADF-STEM images of nanoparticles according to examples and comparative examples.

FIG. 7 shows TEM images of intermediates of nanoparticles according to a comparative example synthesized from core structures, the intermediates obtained by stopping reaction at 5 seconds, 20 seconds, and 60 seconds by using mercaptopropanol.

FIG. 8 shows images and a graph obtained by analyzing results of formation of shell structures according to a ratio of a core structure to a macrocyclic host molecule.

FIG. 9 shows quantified macrocyclic host molecules provided onto a core structure.

FIG. 10 shows graphs of SERS results of nanoparticles bound to a Raman-active material.

FIG. 11 shows an image and a graph showing analysis results of influence of GSH on SERS intensity of nanoparticles.

FIG. 12 shows analysis results of AFM-related Raman spectroscopy and distribution of a SERS enhancement factor (EF).

FIG. 13 shows results of analyzing SERS-based imaging ability of nanoparticles bound to Raman-active materials.

FIG. 14 shows HAADF-STEM image of nanoparticles bound to various types of Raman-active materials on a scale of 100 nm.

FIG. 15 shows SERS-based images of nanoparticles bound to various types of Raman-active materials.

FIG. 16 shows images of HeLa cells overlapped by SERS-based images.

### MODE OF DISCLOSURE

The present invention may have various modifications and alternative forms. Hereinafter, specific embodiment thereof has been shown by way of example in the drawings and will be described in detail below. However, it should be understood that it is not intended to limit the present invention to the particular forms disclosed, but on the contrary, the present invention is to cover all modifications, equivalents, and alternative falling within the spirit and the scope of the disclosure.

According to an embodiment of the present invention, in a nanoparticle having a core-shell structure, a macrocyclic host molecule is provided on the surface of a core structure and a Raman-active material is inserted into the macrocyclic host molecule by non-covalent bonds, and thus nanoparticles having a stable structure and excellent spectroscopic performance may be provided.

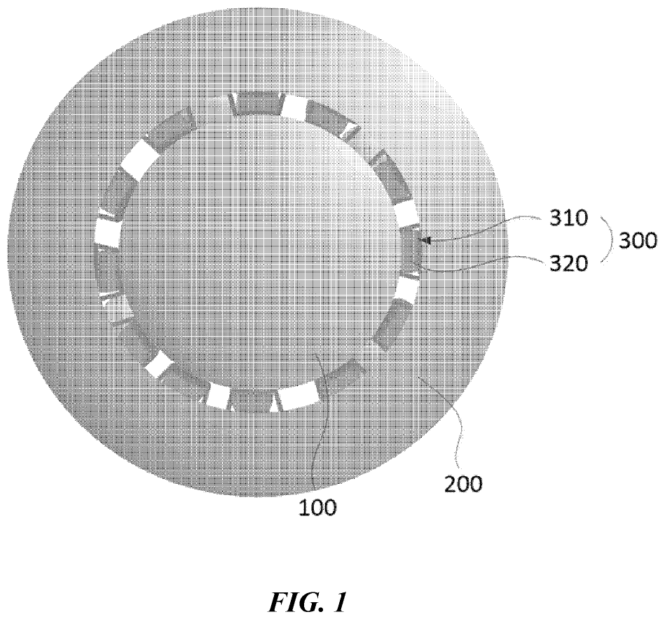

FIG. 1 is a cross-sectional view of a nanoparticle according to an embodiment of the present invention.

Referring to FIG. 1, the nanoparticle includes a core structure 100, and a shell structure covering the core structure 100 and separated from the core structure 100 by a nanogap, wherein a macrocyclic host molecule 310 exhibiting hydrophobicity inside and hydrophilicity outside and a Raman-active material 320 inserted into the macrocyclic host molecule 310 are provided on the surface of the core structure 100, and the macrocyclic host molecule 310 and the Raman-active material 320 fill the nanogap.

The core structure 100 is provided at the center of the nanoparticle and may serve as a base on which the shell structure 200, the macrocyclic host molecule 310, the Raman-active material 320, and the like are provided. The core structure 100 may have a spherical shape, an elliptical shape, a polyhedral shape, (e.g., cube and cuboid), etc.

The core structure 100 may have an average diameter of about 1 nm to about 100 nm. In a core structure 100 having different diameters depending on direction, the average diameter of the core structure 100 may refer to an average of all diameters.

The core structure 100 may include at least one material selected from the group consisting of gold, silver, and copper. If required, the core structure 100 may be formed of gold. Because gold is chemically stable, it is suitable for detection of a substance. In addition, gold has advantages of allowing the macrocyclic host molecule 310 to be easily attached to the surface which will be described below.

The shell structure 200 may be provided to be spaced apart from the core structure 100.

The shell structure 200 is provided in a form covering the core structure 100. The shell structure 200 may have a hollow space such that the core structure 100 is provided therein. A shape of the inner space of the shell structure 200 may correspond to a shape of the core structure 100. For example, when the core structure 100 has a spherical shape, the inner space of the shell structure 200 may also have a spherical shape. In this case, because the shell structure 200 covers the core structure 100, the inner space of the shell structure 200 may have a diameter greater than that of the core structure 100.

An outer circumferential surface of the shell structure 200 may have a shape different from that of the inner space (inner surface) of the shell structure 200. For example, the inner space of the shell structure 200 may have a spherical shape, and the outer circumferential surface may have a polyhedral shape such as a hexahedral shape, an octahedral shape, or the like. While the inner space of the shell structure 200 may vary in shape according to a shape of the core structure 100, the outer circumferential surface of the shell structure 200 may vary in shape according to an aggregation pattern of precursor particles used to form the shell structure 200 and a merged shape of nanobridges formed by aggregation of the precursor particles.

The shell structure 200 may protect the macrocyclic host molecule 310 and the Raman-active material 320 provided on the surface of the core structure 100. Specifically, the macrocyclic host molecule 310 and the Raman-active material 320 may be provided between the core structure 100 and the shell structure 200, and the shell structure 200 prevents the macrocyclic host molecule 310 and the Raman-active material 320 provided therein from being denatured by reaction with a compound outside the nanoparticle. Therefore, detection of a substance may be more stably conducted using the nanoparticle.

The shell structure 200 may include at least one material selected from the group consisting of gold, silver, and copper. If required, the shell structure 200 may be formed of gold. Because gold is chemically stable, it is suitable for detection of a substance.

A nanogap is provided between the shell structure 200 and the core structure 100. The nanogap increases intensities of various optical signals such as Raman signals, fluorescent signals, nonlinear optics, and IR absorption by focusing/constraint of an electromagnetic field amplified therein. Among the signals amplified in the nanogap, the Raman signal is an inelastic scattering signal derived from a vibrational mode of a molecule. Although an intensity of the Raman signal is very weak, the Raman signal is easily distinguished from complex signals and more efficiently used in multiple detection than in detection of a signal having a wide peak such as fluorescence, due to a relatively small half-width. By using a plasmonic nanogap structure, a weak Raman signal may be considerably amplified, and this process is referred to as SERS.

However, due to strong amplification/focusing of an electromagnetic field occurring in the nanogap, an optical signal is sensitively altered even by a small change such as the size or shape of the nanogap and position of molecules. Therefore, a strategy for uniformly and reproducibly synthesizing a very small nanogap with a size of about 1 nm with a high yield and for locating a desired optically active material inside the nanogap is required to reproducibly implement optical signals.

The nanogap may have a size of 0.1 nm to 10 nm. By providing the nanogap having the above-described size, a Raman signal may be amplified. Due to the nanogap, the core structure 100 may be separated from the shell structure 200 without being in contact with each other. In some areas, the core structure 100 may be in contact with the shell structure 200 via nanobridges. Therefore, the “nanogap” used herein does not necessarily mean a space completely separating the core structure 100 from the shell structure 200.

The macrocyclic host molecule 310 and the Raman-active material 320 may be located in the nanogap.

The macrocyclic host molecule 310 is applied to the surface of the core structure 100 and fixes the Raman-active material 320 in the nanogap. The macrocyclic host molecule 310 may be a material having a ring shape and exhibiting hydrophobicity inside and hydrophilicity outside. Thus, the inside of the ring of the macrocyclic host molecule 310 may have hydrophobicity and the outside of the ring may have hydrophilicity. Therefore, after being inserted into the ring of the macrocyclic host molecule 310, a hydrophobic material may form non-covalent bonds with the inside of the ring exhibiting hydrophobicity and may be affected by a repulsive force from the outside the ring exhibiting hydrophilicity. Accordingly, once inserted into the macrocyclic host molecule 310, a substance may be stably fixed therein without being easily escaping therefrom. By using this principle, the Raman-active material 320 may be fixed in the macrocyclic host molecule 310.

The macrocyclic host molecule 310 may include at least one compound selected from the group consisting of cyclodextrin, cucurbituril, calixarene, and pillararene and a derivative thereof. For example, the macrocyclic host molecule 310 may be mono-(6-mercapto-deoxy)-β-cyclodextrin.

The Raman-active material 320 refers to a material facilitates detection and measurement of an analyte by using a Raman detection device when attached to the analyte. The Raman-active material 320 available in Raman spectroscopy includes organic atoms and molecules or inorganic atoms and molecules, etc. Specific examples of the Raman-active material include FAM, Dabcyl, tetramethyl rhodamine-5-isothiocyanate (TRITC), malachite green isothiocyanate (MGITC), X-rhodamine-5-isothiocyanate (XRITC), 3,3-diethylthiadicarbocyanine iodide (DTDC), tetramethyl rhodamine isothiol (TRIT), 7-nitrobenz-2-1,3-diazole (NBD), phthalic acid, terephthalic acid, isophthalic acid, para-aminobenzoic acid, erythrosine, biotin, digoxigenin, 5-carboxy-4′,5′-dichloro-2′,7′-dimethoxy, fluorescein, 5-carboxy-2′,4′,5′,7′-tetrachlorofluorescein, 5-carboxyfluorescein, 5-carboxyrhodamine, 6-carboxyrhodamine, 6-carboxytetramethyl aminophthalocyanine, azomethine, cyanine (Cy3, Cy3.5, and Cy5), xanthine, succinylfluorescein, aminoacridine, quantum dots, carbon allotropes, cyanides, thiol, chlorine, bromine, methyl, phosphor, and sulfur, but are not limited thereto.

The Raman-active material 320 may be a material exhibiting a distinct Raman spectrum and, particularly, capable of binding to or associating with different types of analytes. Preferably, molecules resonating with excitation laser wavelength used in Raman analysis to exhibit a higher intensity of a Raman signal may be used as the Raman-active material 320.

The Raman-active material 320 is provided in a form of inserted into the above-described macrocyclic host molecule 310. For reproducible and strong SERS signals, a strategy of locating the Raman-active material 320 in the nanogap is important as well as uniformly synthesizing the nanogap structure. In addition, various different Raman-active materials 320 should be introduced into the nanogap for multiple detection, and signals therefrom should be distinguishable.

According to an embodiment of the present invention, the macrocyclic host molecule 310 is provided on the surface of the core structure 100, and the Raman-active material 320 is inserted into the macrocyclic host molecule 310. Because the macrocyclic host molecule 310 is uniformly provided, the shell structure 200 covering the macrocyclic host molecule 310 may also be provided in a form including a uniform nanogap. Therefore, the size and uniformity of the nanogap and the position of the Raman-active material 320 which affects the Raman spectrum are guaranteed, and thus the nanoparticle may exhibit a distinct Raman signal.

The structure and composition of the nanoparticle according to an embodiment of the present invention have been described above. Hereinafter, a method for synthesizing a nanoparticle will be described in more detail.

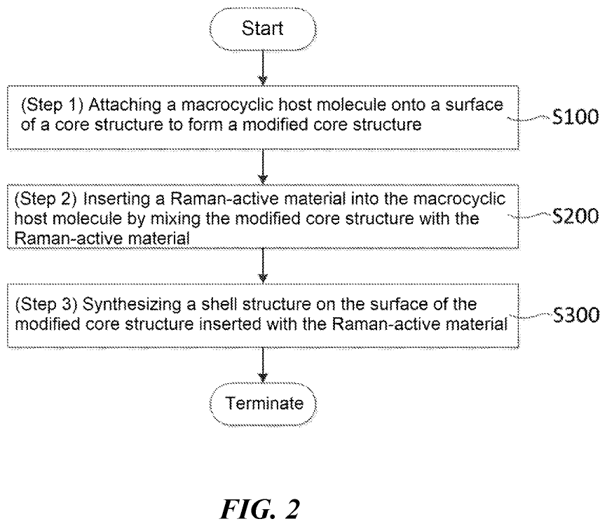

FIG. 2 is a flowchart illustrating a method for synthesizing nanoparticles according to an embodiment of the present invention.

Referring to FIG. 2, the method for synthesizing nanoparticles includes a first step of attaching a macrocyclic host molecule onto a surface of a core structure to form a modified core structure (S100), a second step of inserting a Raman-active material into the macrocyclic host molecule by mixing the modified core structure with the Raman-active material (S200), and a third step of synthesizing a shell structure on the surface of the modified core structure inserted with the Raman-active material (S300), wherein a nanogap filled with the macrocyclic host molecule and the Raman-active material is provided between the core structure and the shell structure.

First, the first step (S100) may be performed by preparing the core structure, mixing the prepared core structure with the macrocyclic host molecule, and incubating the mixture. In addition, the first step (S100) may further include a step of providing a core structure capped with cetyltrimethylammonium bromide (CTAB); and a step of providing a core structure capped with cetyltrimethylammonium chloride (CTAC) by reacting the CTAB-capped core structure with L-ascorbic acid and HAuCl₄. By performing the first step (S100) including the above-described steps, the macrocyclic host molecule may be uniformly attached onto the core structure. Therefore, a nanoparticle having a uniformly formed nanogap may be formed in the following process.

Subsequently, the second step (S200) is a step of inserting the Raman-active material into the macrocyclic host molecule by mixing the modified core structure with the Raman-active material. In the second step (S200), the macrocyclic host molecule binds to the Raman-active material via non-covalent bonds on the modified core structure.

According to the related art, in order to provide a Raman-active material to a core structure, the Raman-active material is bound to a substance such as an oligonucleotide, and then the oligonucleotide linked to the Raman-active material is bound to the core structure. However, this method has problems in that a production method is very complicated and a production yield thereof is low because the oligonucleotide-Raman-active material complex formed by covalent bonds should be individually synthesized.

According to the related art, an oligonucleotide-Raman-active material complex is not produced with a yield of 100% although the oligonucleotide is reacted with the Raman-active material, and it is difficult to selectively separate only the oligonucleotide-Raman-active material complex from a result of reaction, and thus there was a problem that many oligonucleotides without the Raman-active material could be bound to the core structure during a subsequent process of modifying the surface of the core structure with the oligonucleotide-Raman-active material complex. Such non-uniform modification and binding are obstacles to generating reproducible and strong SERS signals.

According to an embodiment of the present invention, the macrocyclic host molecule may be easily bound to the Raman-active material by non-covalent bonds with a high yield in a reactor. Unlike the related art in which the reaction occurs by individual covalent bonds, a macrocyclic host molecule-Raman-active material conjugate may be provided with a high yield relatively easily. Also, in the second step (S200), more uniform modification and insertion of the Raman-active material may be obtained by attaching the macrocyclic host molecule to the core structure and then the Raman-active material is inserted into the macrocyclic host molecule compared to the related art in which the oligonucleotide bound to the Raman-active material is attached to the core structure.

Then, in the third step (S300), the shell structure is synthesized on the surface of the modified core structure inserted with the Raman-active material. In this case, since the shell structure is formed on the macrocyclic host molecule uniformly attached to the surface of the core structure, a nanogap may also be uniformly formed between the core structure and the shell structure.

In the third step (S300), the shell structure may be formed as nanobridges growing by aggregation of precursor particles merge.

The method of synthesizing the nanoparticles according to an embodiment of the present invention is described above. Hereinafter, superior effects of the nanoparticles according to the present invention will be described in more detail with reference to data of examples and comparative examples.

According to an embodiment, cyclodextrin (CD)-based plasmonic nanogap particles (CIPs) were designed and synthesized by using cyclodextrin as interlayer molecules promoting formation inner nanogaps while an Au shell structure grows on an Au core structure. In this case, various Raman-active materials were used as guest molecules and cyclodextrin was used as host molecules. The synthesized nanoparticles had uniform nanogaps having a size of about 1 nm and formed in the particles and a yield was about 97%. The nanoparticles generate strong and stable SERS signals with strong SERS enhancement factor (EF) values in a narrow range of distribution. In addition, 10 different types of the Raman-active material were able to be simply introduced thereinto by adding a dye solution before the Au shell structure grew, and 10 unique SERF spectra could be obtained in the same solution, and thus the possibility of multiple detection was confirmed.

Experimental Example 1. Synthesis of Au Core-Shell Nanogap Nanoparticle

In order to synthesize Au core-shell nanogap nanoparticles, materials were prepared as described below.

L-ascorbic acid (AA), gold (III) chloride (HAuCl₄·3H₂O), crystal violet (CV), rhodamine B (RB), methylene blue (MB), safranin O (SO), basic fuchsin (BF), brilliant blue G (BBG), Nile Blue A (NBA), bromophenol blue (BPB), L-glutathione (reduced), sodium dodecyl sulfate (SDS), cetyltrimethylammonium bromide (CTAB), N-(3-dimethylamino propyl)-N′-ethylcarbodiimide hydrochloride (EDO), N-hydroxysulfosuccinimide sodium salt (Sulfo-NHS), and 2-(N-morpholino)ethanesulfonic acid (MES) were purchased from Sigma-Aldrich. Ethidium bromide (EtBr) and Pyronin Y (PYY) were purchased from Thermo Fisher Scientific. Mono-(6-mercapto-6-deoxy)-β-cyclodextrin (CD) was purchased from Zhiyuan Biotechnology. Sodium borohydride was purchased from Alfa Aesar. Cetyltrimethylammonium chloride (CTAC) was purchased from Tokyo Chemical Industry (TCI). Cyclo(Arg-Gly-Asp-D-Phe-Lys) (cRGDyK, cRGD) peptides were purchased from Peptides International, Inc. (Louisville, KY, USA). Carboxymethyl-PEG-thiol (CM-PEG-SH, Mw≈5000) was purchased from Laysan Bio, Inc. (Arab, AL, USA). N,N-dimethylformamide (DMF) and dimethyl sulfoxide (DMSO) were purchased from Samchun Chemicals. Deionized water (DIW; Milli-Q,>18.0 MΩ) was used for all experiments. All chemicals were used without further purification.

### Experimental Example 1-1. Synthesis of 50 nm Au Core Structure (AuNS)

A CTAC-capped Au core structure (AuNS) having a size of 50 nm was synthesized according to the following procedure. First, CTAB-capped Au clusters were prepared. 600 μL of a cold 10 mM NaBH₄ solution was quickly added to 10 mL of an aqueous solution of HAuCl₄ (0.25 mM) and CTAB (100 mM) and stirred (at 500 rpm). For complete decomposition of NaBH₄ in the mixture, the mixture was maintained at 27° C. for 2 hours. Subsequently, an HAuCl₄ aqueous solution (0.5 mM, 2 mL) and CTAB-capped Au clusters (50 μL) were added to an aqueous solution containing CTAC (200 mM, 2 mL) and L-ascorbic acid (AA, 100 mM, and 1.5 mL) by one-shot injection to synthesize CTAC-capped AuNS with a size of 10 nm or less. The reaction was continued at 27° C. for 15 minutes. A product was collected by centrifugation at 15000 rpm for 30 minutes and washed twice with a 1 mM CTAC solution, and then finally dispersed in 1 mL of a 20 mM CTAC aqueous solution. Finally, the HAuCl₄ aqueous solution (0.5 mM, 2 mL) was added to the aqueous solution containing CTAC (100 mM, 2 mL), AA (8 mM, 130 μL) and 10 nm AuNS (6 μL) using a syringe pump while stirring to synthesize CTAC-capped 50 nm AuNS. Upon completion of the injection, a product was placed at 27° C. for 10 minutes. A product was collected by centrifugation and washed with an SDS solution and a CTAC solution.

### Experimental Example 1-2. Synthesis of Cyclodextrin-Modified 50 nm Au Core Structure (CD-AuNS)

In order to prepare CD-AuNS nanospheres, mono-(6-mercapto-6-deoxy)-β-cyclodextrin (CD) was attached to the surface of the 50 nm AuNS. 10 μL of 50 mM CD dissolved in DMSO was mixed with 1 mL of 100 pM AuNS in a 0.1% SDS solution. The solution was incubated at 60° C. for 18 hours. A product solution was collected by centrifugation (at 7500 rpm for 3 minutes) and dispersed three times in a 0.1% SDS solution, and then finally dispersed in a SDS solution for future use.

### Experimental Example 1-3. Synthesis of Cyclodextrin-Based Nanoparticle Having Inner Nanogap

Cyclodextrin-based intra-nanogap particles (CIP) were synthesized by forming an Au shell structure on each of the CD-AuNS nanospheres in the presence of a Raman-active material. To this end, first, 100 μL of a 50 pM CD-AuNS solution was mixed with 20 μL of a 100 mM CTAC solution. Then, 2 μL of 10 mM crystal violet (CV) dissolved in DMF was added thereto and mixed therewith. Subsequently, 25 μL of a 40 mM L-ascorbic acid (AA) solution and 25 μL of 5 mM HAuCl₄ were sequentially added to the mixture.

A produced mixture was incubated at room temperature for 30 minutes. A product was collected by centrifugation (at 6000 rpm for 3 minutes) and washed four times with a 1 mM CTAC solution and dispersed in 50 μL of a 1 mM CTAC solution. Subsequently, desorption of the Raman-active material adsorbed onto the surface was induced by adding 5 μL of a 1 mM glutathione solution to 50 μL of a 100 pM nanoparticle solution. All SERS measurements were performed after treatment with glutathione to exclude influence of the Raman-active material adsorbed to the surface. Nanoparticles including the other Raman-active materials were also synthesized in the same manner while varying volumes and solvents of Raman-active material solutions. Volumes and solvents of the Raman-active materials used in synthesis of each of the nanoparticles are shown in Table 1 below.

TABLE 1

| Raman-active | | |
|---|---|---|
| material (10 mM) | Solvent | Volume (μL) |
| CV | DMF | 2 |
| NBA | DMF | 2 |
| BF | DMF | 2 |
| SO | DMF | 10 |
| EtBr | DIW | 10 |
| PYY | DMF | 20 |
| MB | DMF | 20 |
| BBG | DMF | 20 |
| RB | DMF | 20 |
| BPB | DMSO | 20 |

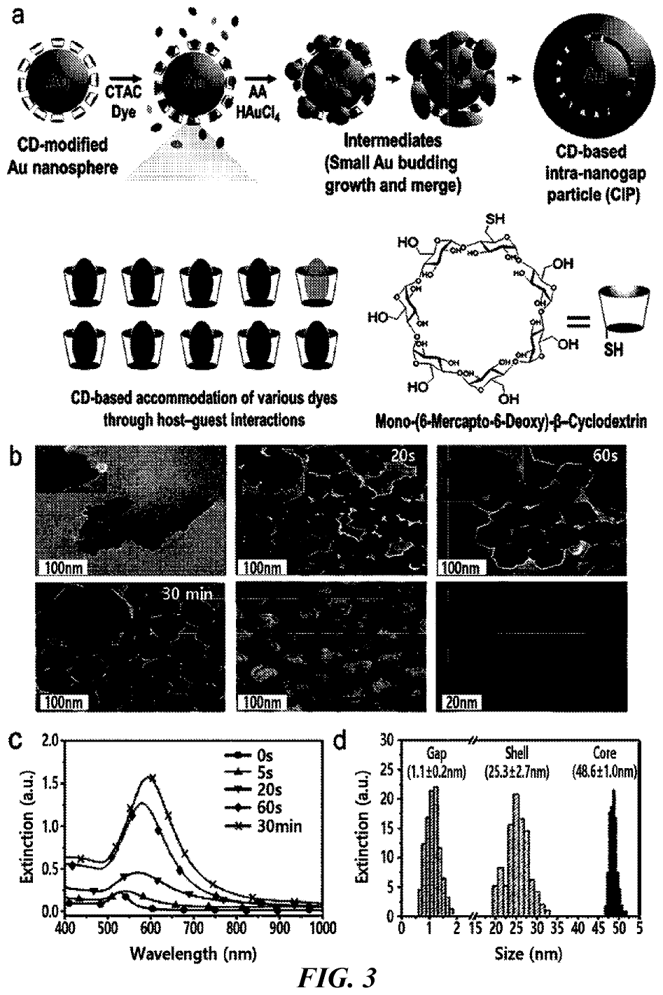

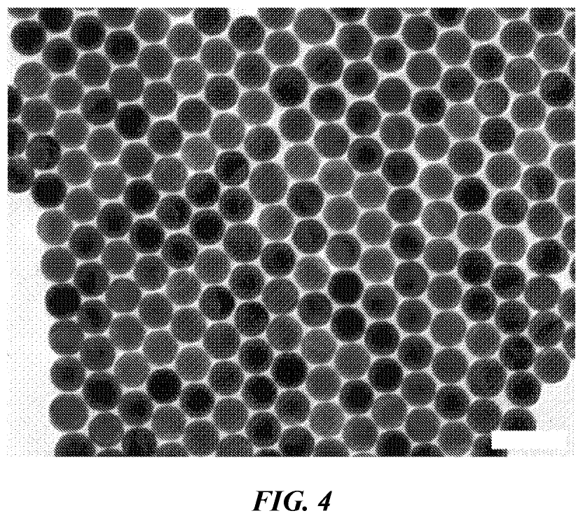

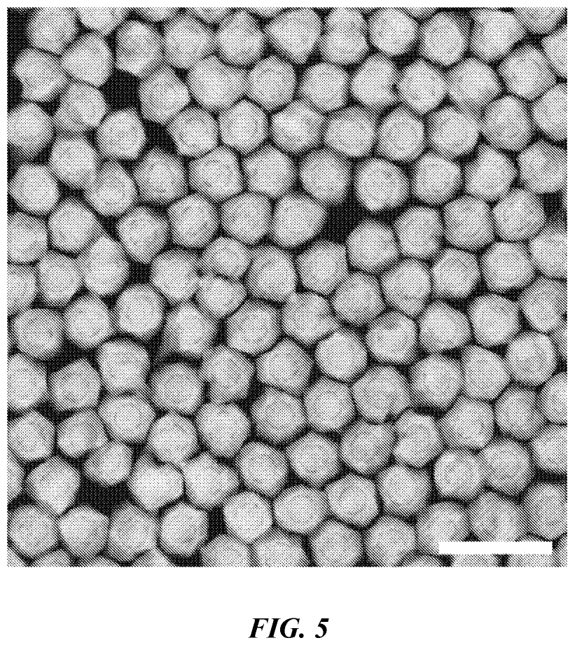

Hereinafter, analysis results of structures and physical properties of the synthesized nanoparticle and intermediates will be described with respect to Experimental Examples 1-1 to 1-3. FIG. 3 shows graphs and images illustrating synthesis of macrocyclic host molecule-based nanoparticles according to an embodiment of the present invention and analysis results thereof. FIG. 4 is a TEM image of core structures having a size of 50 nm on a scale of 100 nm. FIG. 5 is an HAADF-STEM image of nanoparticles on a scale of 200 nm.

(A) FIG. 3 schematically shows synthesis of a nanoparticle including various Raman-active materials in a nanogap. (B) of FIG. 3 shows magnified TEM images of structures of intermediates after 5 seconds, 20 seconds, and 60 seconds, respectively, from addition of ascorbic acid (AA) and HAuCl₄. (C) of FIG. 3 shows a UV-vis spectrum of intermediates and nanoparticles, and (d) of FIG. 3 shows size distribution of nanogaps, shell structures, and core structures of nanoparticles.

Referring to (a) of FIG. 3 and FIG. 4, as described above, a method of forming an Au shell structure on an Au core structure modified with mono-(6-mercapto-6-deoxy)-β-cyclodextrin was initiated by introducing L-ascorbic acid (AA) into a solution containing the core structures, cetyltrimethylammonium chloride (CTAC), and crystal violet (CV). A material produced thereby was CV-coated nanoparticles (CV-CIPs).

Referring to (b) of FIG. 3, as shown in the transmission electron microscope (TEM) image, small Au budding structures grow from the surface of the core structure and merge to form a continuous shell structure while an inner gap is formed inside the shell structure at the same time.

Referring to (c) of FIG. 3, structural changes while the reaction proceeds may be identified by UV-vis spectrum.

Additionally, referring to (d) of FIG. 3 and FIG. 5, in the high-angle annular dark-field scanning transmission electron microscope (HAAF-STEM) image, uniform formation of inner nanogaps of about 1 nm was observed in 97% or more of the structures.

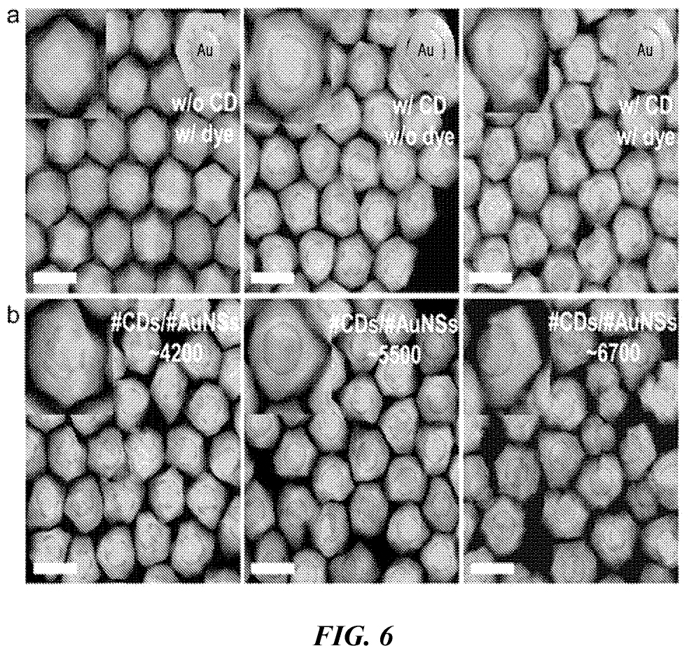

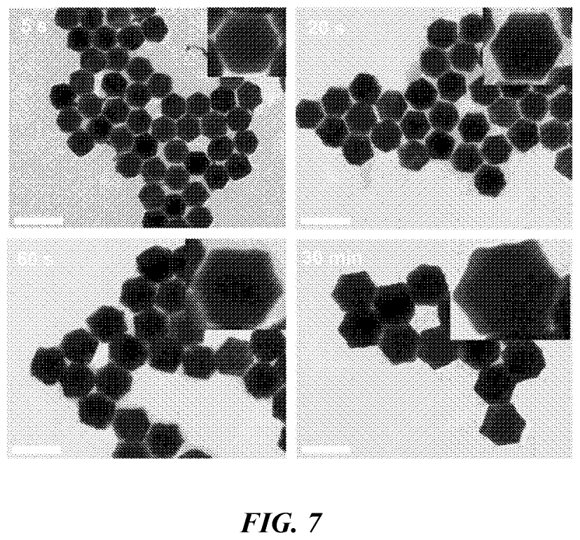

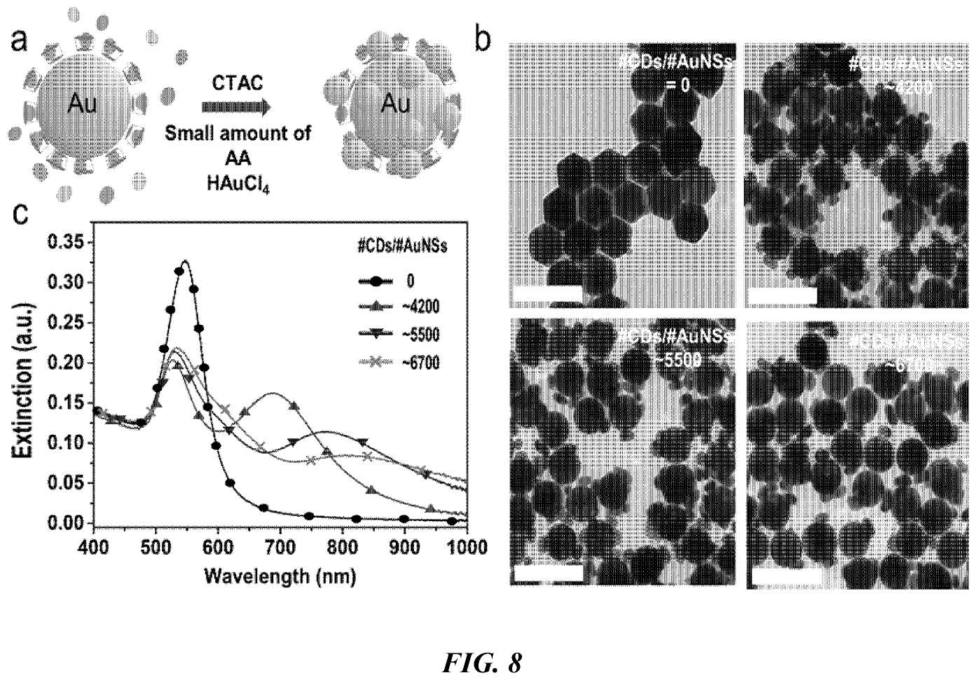

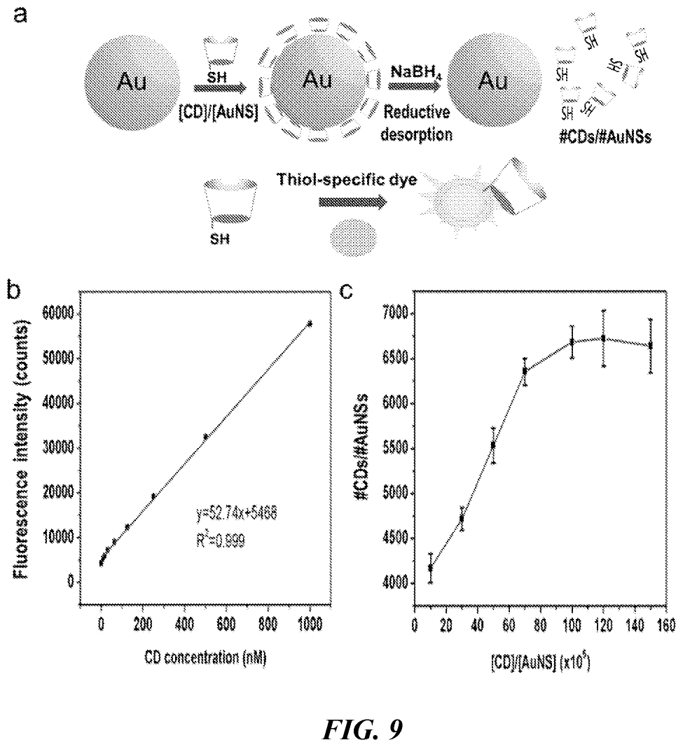

FIG. 6 shows HAADF-STEM images of nanoparticles according to examples and comparative examples. FIG. 7 shows TEM images of intermediates of nanoparticles according to a comparative example synthesized from core structures, the intermediates obtained by stopping reaction at 5 seconds, 20 seconds, and 60 seconds by using mercaptopropanol. FIG. 8 shows images and a graph obtained by analyzing results of formation of shell structures according to a ratio of core structures to macrocyclic host molecules. FIG. 9 shows quantified macrocyclic host molecules provided onto core structures.

(A) of FIG. 6 shows an image of nanoparticles synthesized using only a Raman-active material without cyclodextrin (left), an image of nanoparticles synthesized using only cyclodextrin without the Raman-active material (middle), and an image of nanoparticles synthesized using both the cyclodextrin and the Raman-active material. In the middle and right images, a cyclodextrin (CD)/AuNS ratio was fixed to 5500. (B) of FIG. 6 shows nanoparticles synthesized while changing the cyclodextrin (CD)/AuNS ratio (about 4200 (left), about 5500 (middle), and about 6700 (right)). Each of the images was magnified on a scale of 100 nm.

Referring to the left image of (a) of FIG. 6 and FIG. 7, it was confirmed that inner gaps were not formed when the core structures were used without being modified with cyclodextrin. Additionally, referring to the middle image of (a) of FIG. 6, the nanogaps were clearly observed when the nanoparticles were synthesized without the Raman-active material, and this indicates that cyclodextrin on the core structures affected formation of the nanogaps. Referring to the right image of (a) of FIG. 6, the inner nanogaps were observed in the nanoparticles in the case of using both cyclodextrin and the Raman-active material.

Referring to (b) of FIG. 6 and FIG. 8, it may be confirmed that CD-AuNSs having different numbers of CD molecules may be obtained by varying a concentration of cyclodextrin (CD) to the core structure (AuNS). As the CD/AuNS ratio increases, the number of the budding structures of the core decreased. This indicates that the surface of the CD-modified core structure suppresses formation of budding structures ((a), (b), and (c) of FIG. 8).

The change in the CD/AuNS ratio affected the shape of nanoparticles. At a low CD/AuNS of about 4200, a greater bridging structure was observed between the core structure and the shell structure. At a high CD/AuNS ratio of about 6700, the shell structure was not formed around the core structure in many particles and low structural uniformity was observed (See (b) of FIG. 6).

At a CD/AuNS of about 5500, inner nanogaps were clearly and uniformly formed in 97% or more of particles and complete shell structures were formed.

Next, referring to FIG. 9, cyclodextrin (CD) molecules in the core structures (AuNS) having a size of 50 nm were quantified. The number of cyclodextrin provided to each core was quantified by a thiol-selective dye-based fluorescence spectroscopy.

In order to quantify the number of cyclodextrin molecules attached to the surfaces of the core structures (AuNS), 55 μL of a 100 mM NaBH₄ solution was added to 55 μL of a 150 pM cyclodextrin modified core structure (CD-AuNS) solution. After incubating the mixture at room temperature for 5 minutes, reductive desorption of adsorbed thiolate CD molecules was performed. Free thiolate CD molecules were collected in a supernatant solution after centrifugation. Then, the solution was incubated at room temperature for one day to decompose NaBH₄ in the collected solution. Then, 30 μL of a free thiol CD molecule solution was mixed with an equal volume of a thiol selective dye solution for 5 minutes. Fluorescence signals generated from the mixture were obtained at an excitation wavelength of 380 nm and an emission wavelength of 510 nm. The number of CD molecules on the surface of the core structures was obtained from standard concentration curves obtained in the same procedure using CD solutions having various concentrations.

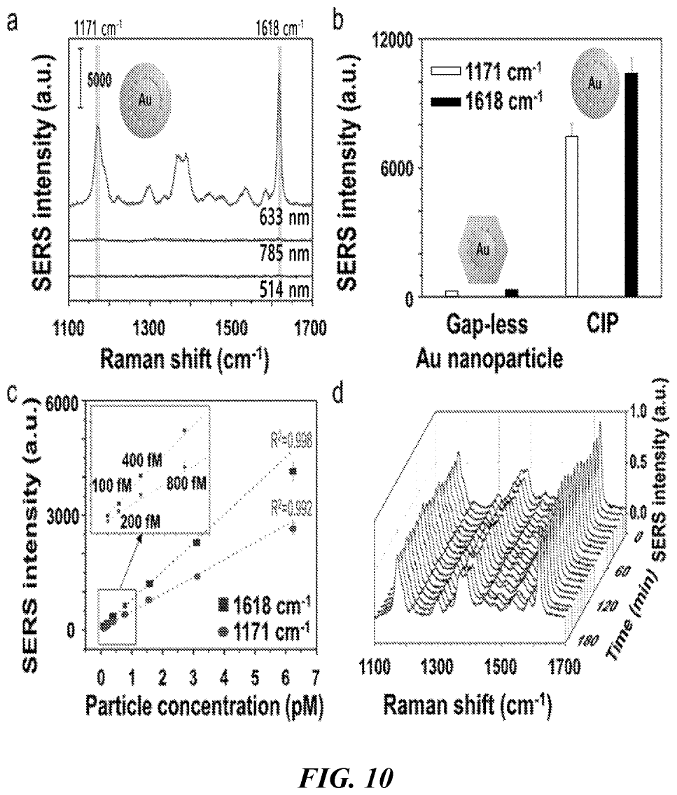

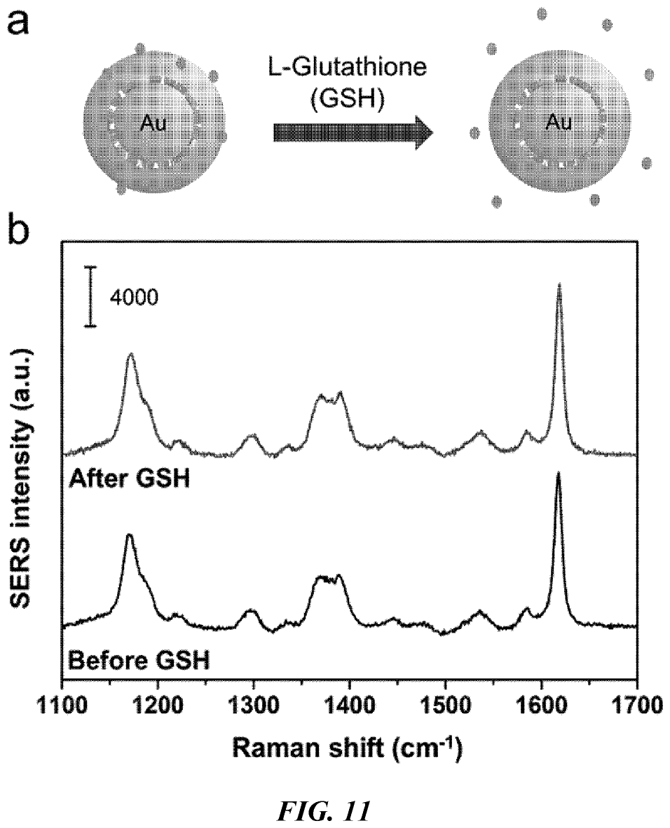

FIG. 10 shows graphs of SERS results of nanoparticles bound to a Raman-active material. FIG. 11 shows an image and a graph showing analysis results of influence of GSH on SERS intensity of nanoparticles. FIGS. 10 and 11 show results of SERS signals measured using nanoparticles including a CV dye.

(A) of FIG. 10 is a wavelength-dependent SERS spectrum and excitation wavelengths are 633 nm (3 mW), 785 nm (6 mW), and 514 nm (5 mW) and acquisition time is 10 seconds. Red and blue lines indicate two different peaks at 1171 cm⁻¹ and 1618 cm⁻¹. (B) of FIG. 10 is a comparison result of SERS intensity between two different peaks of the nanoparticles synthesized without the cyclodextrin-modified core structure. (C) of FIG. 10 shows changes in SERS intensity depending on the concentration of nanoparticles at two different peaks, and (d) of FIG. 10 shows a time-dependent SERS spectrum when continuously exposed to a laser (633 nm, 3 mW) for 3 hours

Referring to (a) of FIG. 10, incidence excitation sources of 514 nm, 633 nm and 785 nm were used to investigate an optical excitation wavelength. Among them, a 633 nm laser generated the strongest and most distinctive wavelength. An extinction maximum and a maximum CV absorption of the nanoparticles exhibited lower spectral overlaps in the case of using the 633 nm laser compared to the lasers using the other wavelengths. Therefore, the 633 nm laser was selected to collect SERS signals.

Before measuring the SERS signals, desorption of the dye from the surfaces of the nanoparticles was induced by adding glutathione to a nanoparticle solution. A SERS spectrum of the nanoparticle solution was hardly affected by glutathione treatment indicating that the dye (Raman-active material) inside the nanogap exhibits a SERS signal and is well protected by the shell structure (See FIG. 11).

Referring to (b) of FIG. 10, the SERS intensity increased by about 28 times and 32 times, respectively at 1171 cm⁻¹ and 1618 cm⁻¹, compared to gapless Au-Au core-shell nanoparticles formed without cyclodextrin.

Referring to (c) of FIG. 10, a linear relationship between a concentration of nanoparticles and a SRES intensity was obtained and a femtomolar concentration was detected. An important point is that SERS signals were stably obtained from the nanoparticle solution without a visible change in signals although the solution was continuously exposed to a laser, ((d) of FIG. 10), which indicates that SERS signals may be stably and quantitatively obtained from nanoparticles.

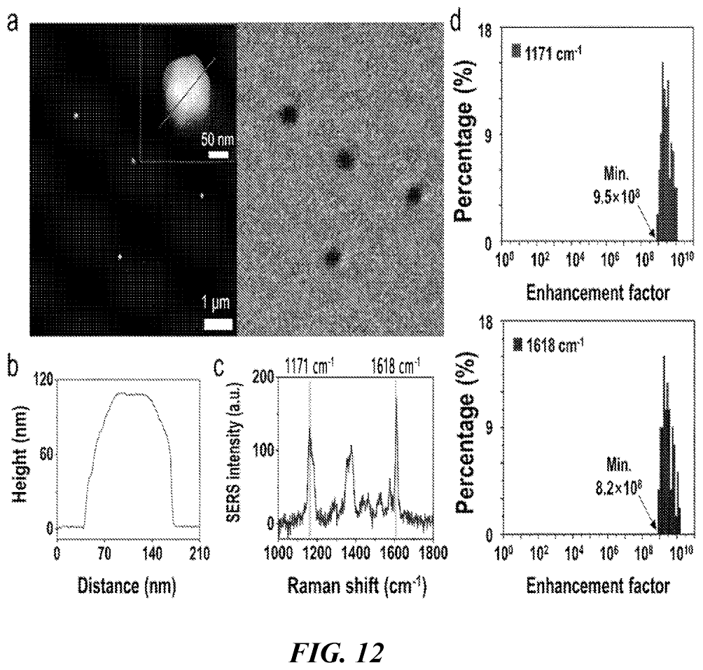

FIG. 12 shows analysis results of AFM-related Raman spectroscopy and distribution of a SERS enhancement factor (EF).

(A) of FIG. 12 shows an AFM image (left) and a Rayleigh scattering image (right) of nanoparticles to collect a SERS spectrum from individual particles. (B) of FIG. 12 is a height profile across a nanoparticle along a red line of (a) of FIG. 12. (C) of FIG. 12 is a representative SERS spectrum of a single CV-nanoparticle. Red and blue lines indicate two different peaks at 1171 cm⁻¹ and 1618 cm⁻¹, respectively. (D) of FIG. 12 shows distribution of SERS EF with respect to two fingerprint peaks.

Single-particle SERS analysis was conducted on nanoparticles by atomic force microscopy (AFM)-related Raman spectroscopy. For the AFM-related Raman spectroscopy, a sample was prepared on a cover glass by drop-casting 2.5 pM nanoparticles in a 0.1 mM CTAC solution and incubated at room temperature for 3 minutes. The solution remaining on the cover glass was blown off by using an air pump. A SERS spectrum was obtained from nanoparticles in a single particle level by using an AMF-related Raman microscope (Ntegra, NT-MDT) equipped with an inverted microscope (IX73, Olympus) and an oil immersion microscope objective lens (100 X, NA=1.4, Olympus).

A position of an AFM tip and a focus of the laser were matched based on documents of the related art. In general, laser beams were focused on the top surface of the cover glass on which the nanoparticles were positioned. Subsequently, the AFM tip was scanned around a focal point using a piezoelectric x and y tube scanner of an AFM head while a Raman signal of silicon (520 cm⁻¹) of the AFM tip was detected by a CCD (Peltier cooling at—70° C.).

The highest intensity of the Raman signal was observed at the focus of the laser. After the process of tip matching, Rayleigh scattering of individual nanoparticles was obtained using a 633 nm laser and detected by photomultiplier tube. A SERS spectrum of individual nanoparticles was obtained at a position where the Rayleigh scattering occurred in a photomultiplier tube image. The SERS spectrum was obtained using 633 nm (30 μW) with an acquisition time of 2 seconds.

Referring to (a), (b), and (c) of FIG. 12, it was confirmed that the obtained SERS spectrum was generated from single nanoparticles based on Rayleigh scattering and the AFM image of the particles.

Also, referring to (d) of FIG. 12, distribution of the SERS enhancement factor (EF) of single nanoparticles was calculated in 133 particles using peak intensities at 1171 cm⁻¹ and 1618 cm−1. An average EF value of the measured particles was 3.0×10⁹, and the SERS EF of 10⁷ to 10⁸ may be strong enough for single molecule detection. The EF values were observed in first and second distribution ranges of 9.5×10⁸ to 1.2×10¹⁰ and 8.2×10⁸ to 1.5×10¹⁰, respectively, at the peak intensities of 1171 cm⁻¹ and 1618 cm⁻¹. The EF values at 1171 cm⁻¹ were distributed in the very narrow first range of 9.5×10⁸ to 9.5×10⁹ in about 95% of the measured nanoparticles, indicating that the nanoparticle is a highly quantitative SERS probe.

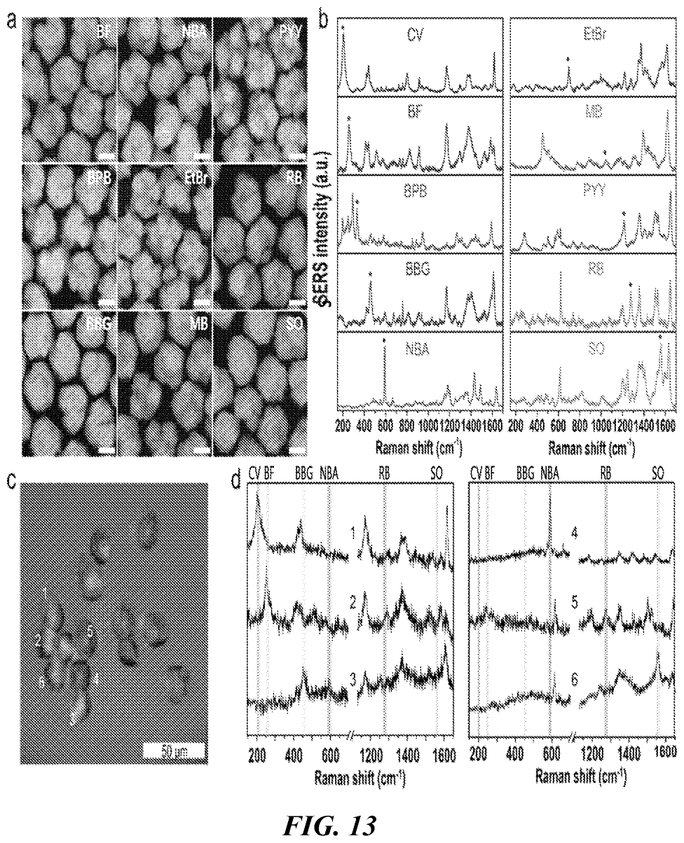

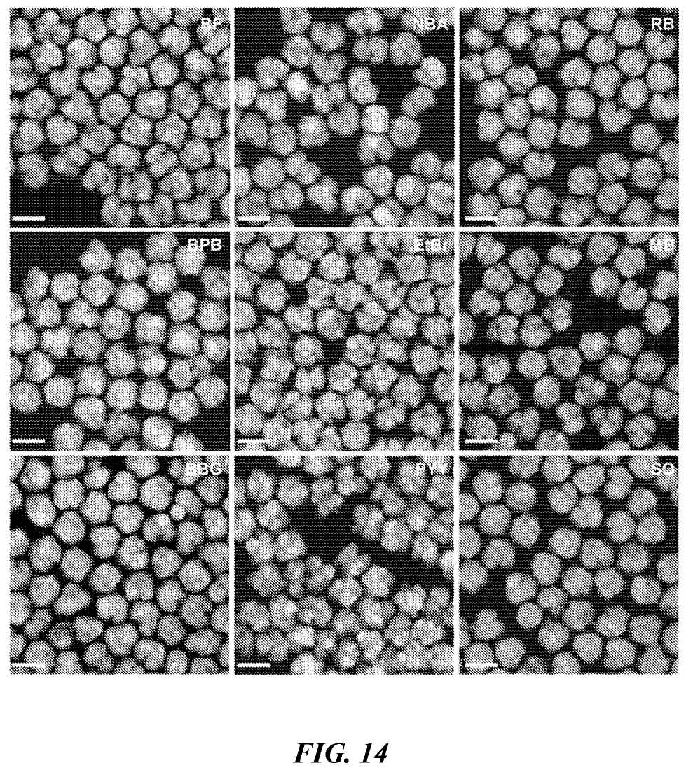

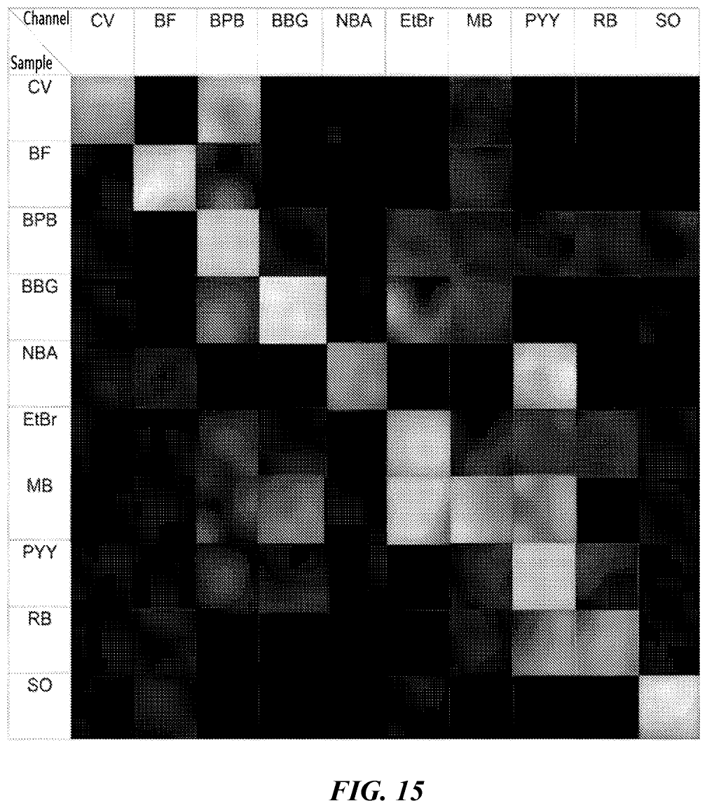

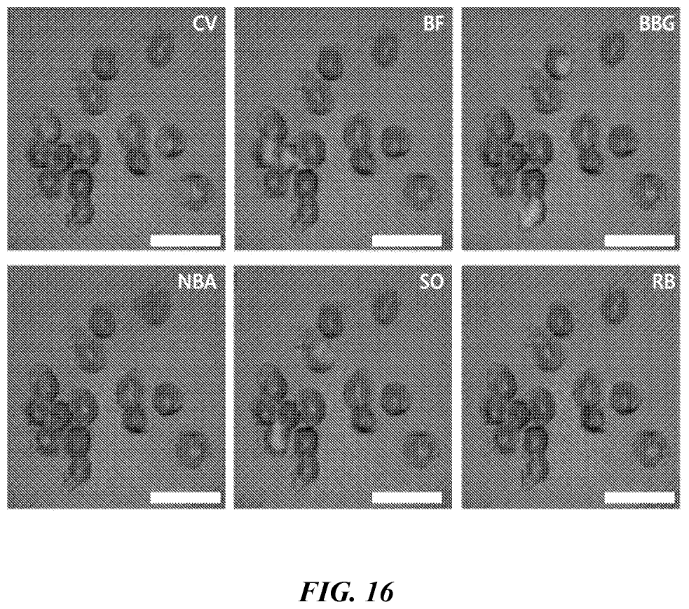

FIG. 13 shows results of analyzing SERS-based imaging ability of nanoparticles bound to Raman-active materials. FIG. 14 shows HAADF-STEM images of nanoparticles bound to various types of Raman-active materials on a scale of 100 nm. FIG. 15 shows SERS-based images of nanoparticles bound to various types of Raman-active materials. FIG. 16 shows images of HeLa cells overlapped by SERS-based images.

Cyclodextrin (CD) may form inclusion complexes with various guest molecules via host-guest interactions, and thus may include various Raman-active materials for multiple detection. 10 different types of Raman-active materials that can for inclusion complexes with cyclodextrin in nanoparticles were introduced.

SERS-based multiple cell imaging was performed as follows. First, before performing SERS-based multiple cell imaging, spectral overlaps were investigated among the 10 fingerprint SERS peaks in the SERS-based imaging (Table 2).

TABLE 2

| Raman-active material | SERS peak (cm⁻¹) |
|---|---|
| CV | 205 |
| BF | 254 |
| BPB | 334 |
| BBG | 458 |
| NBA | 592 |
| ETBR | 698 |
| MB | 1045 |
| PYY | 1215 |
| RB | 1278 |
| SO | 1560 |

To select Raman-active materials with little spectral overlaps, nanoparticles coded with 10 different Raman-active materials were dropped on a cover glass at 10 different positions and dried. Subsequently, SERS-based imaging was conducted on each ring region of 10 coffee-rings in which the nanoparticles were densely disposed. 10 different SERS images were visualized by 10 fingerprint SERS peak channels coded with 10 colors (See Table 2 and FIG. 15). Among the 10 types of the Raman-active materials, 6 types of Raman-active materials (CV, BF, BBG, NBA, RB, and SO), in which little spectral overlaps of the fingerprint SERS peaks was observed, were selected. Finally, SERS-based multiple imaging of HeLa cells was performed using 6 Raman-active material-coded nanoparticles. All SERS measurements were performed using a Raman microscope (Renishaw) with a 633 nm excitation laser of (3 mW) using a 20× objective lens (NA=0.40, Leica), and an acquisition time per each pixel of 5 μm×5 μm was 1 second. Intensities of integrated signals of the 6 fingerprint SERS peaks were coded with 6 colors (See FIG. 16). The merged SERS image was also displayed ((c) of FIG. 13). Referring to (a) of FIG. 13 and FIG. 14, the inner nanogaps were clearly formed in the 10 different types of the Raman-active materials. In this case, formation of the shell structures was affected by the types of the guest Raman-active materials. This indicates that the Raman-active material may be used to adjust the nanogap structure.

Referring to (b) of FIG. 13, it was confirmed that the fingerprint peaks of the 10 different types of Raman-active materials were detected and distinguished from each other by using the single excitation source (633 nm). In this regard, the 6 Raman-active materials (CV, BF, BBG, NBA, RB, and SO) were selected and, referring to FIG. 15, little spectral overlaps were observed between the Raman-active materials.

To investigate the multiple imaging of the nanoparticles, SERS-based multiple imaging of HeLa cells was performed using the nanoparticles coded with the 6 Raman-active materials. The surfaces of the nanoparticles were functionalized with cyclo(Arg-Gly-Asp-D-Phe-Lys) peptides and induce efficient accumulation in HeLa cells due to specific binding affinity to integrin αvβ3 over-expressed by tumor endothelium. The cells of each well were labeled with one of the 6 types of nanoparticles and all wells were combined after labeling.

Referring to (c) of FIG. 13 and FIG. 16, each of 6 types of cells labeled with the nanoparticles respectively coded with the 6 types of different Raman-active materials was individually identified via channels of the 6 different SERS fingerprint peaks minimally overlapping. Therefore, multiplexing function of nanoparticles using a single excitation source was confirmed.

Referring to (d) of FIG. 13, SERS spectra of other cells were also analyzed and almost no spectral overlap was observed in each of the SERS fingerprint peaks. In the case of using a fluorescence spectrum, it is difficult to achieve such multiplexing function and resolution due to wide and simple spectral properties. Therefore, particular tools or analysis methods are required in order to expand the number of available colors using fluorescence. In addition, in the case of using a single laser source, it is very difficult to obtain an image multiplexed with fluorescence signals by using a single excitation source due to a large spectral overlap between different phosphors.

The nanoparticles according to the present invention enable multiple detection and imaging by using SERS. This is because the Raman-active material exhibits several sharp peaks without spectral overlaps by using single wavelength excitation. The synthesis strategy by using cyclodextrin on the core structure showed the possibility of quantitatively locating various Raman-active materials in the nanogap using host-guest interactions without performing complex conjugation and purification processes. Also, it was confirmed that the located Raman-active material was stably protected by the shell structure.

As described above, the present disclosure has been described with reference to particular embodiments, but it is to be understood that the present disclosure is not limited to the above-described embodiments, and various modifications and variations may be made thereto by those skilled in the art in the field of the present disclosure.

Accordingly, the spirit of the present disclosure should not be construed as being limited to the embodiments described, and all the equivalents or equivalents of the claims, as well as the following claims, fall within the scope of the present disclosure.

1. A nanoparticle comprising:
a core structure; and
a shell structure covering the core structure and separated from the core structure by a nanogap,
wherein a macrocyclic host molecule exhibiting hydrophobicity inside and hydrophilicity outside and a Raman-active material inserted into the macrocyclic host molecule are provided on a surface of the core structure, and
the macrocyclic host molecule and the Raman-active material fill the nanogap.

2. The nanoparticle according to claim 1, wherein the macrocyclic host molecule comprises at least one compound selected from the group consisting of cyclodextrin, cucurbituril, calixarene, and pillararene and a derivative thereof.

3. The nanoparticle according to claim 1, wherein the macrocyclic host molecule binds to the Raman-active material by a non-covalent bond.

4. The nanoparticle according to claim 1, wherein the core structure and the shell structure comprise at least one material selected from gold, silver, and copper.

5. The nanoparticle according to claim 1, wherein the nanogap has a size of 0.1 nm to 10 nm.

6. A method for synthesizing a nanoparticle, the method comprising:
a first step of attaching a macrocyclic host molecule onto a surface of a core structure to form a modified core structure;
a second step of inserting a Raman-active material into the macrocyclic host molecule by mixing the modified core structure with the Raman-active material; and
a third step of synthesizing a shell structure on the surface of the modified core structure inserted with the Raman-active material,
wherein a nanogap filled with the macrocyclic host molecule and the Raman-active material is provided between the core structure and the shell structure.

7. The method according to claim 6, wherein the first step is performed by mixing the core structure with the macrocyclic host molecule and incubating the mixture, and
the macrocyclic host molecule comprises at least one compound selected from the group consisting of cyclodextrin, cucurbituril, calixarene, and pillararene and a derivative thereof.

8. The method according to claim 6, wherein the first step further comprises:
a step of providing a core structure capped with cetyltrimethylammonium bromide (CTAB); and
a step of providing a core structure capped with cetyltrimethylammonium chloride (CTAC) by reacting the CTAB-capped core structure with L-ascorbic acid and HAuCl₄,
wherein the macrocyclic host molecule is attached onto the CTAC-capped core structure to form the modified core structure.
